(19) United States
(12) Patent Application Publication
(10) Pub. No.: US 2021/0244822 A1
(43) Pub. Date: Aug. 12, 2021

(54) CONJUGATES WITH INTERNAL AND TERMINAL-END HEPAROSAN LINKAGES

(71) Applicant: The Board of Regents of the University of Oklahoma, Oklahoma City, OK (US)

(72) Inventor: Paul L. DeAngelis, Edmond, OK (US)

(21) Appl. No.: 17/251,147

(22) PCT Filed: Jun. 21, 2019
(86) PCT No.: PCT/US2019/038533

§ 371 Date: Dec. 10, 2020

Related U.S. Application Data
(60) Provisional application No. 62/689,582, filed on Jun. 25, 2018.
(60) Provisional application No. 62/689,553, filed on Jun. 25, 2018.

(51) Int. Cl. A61K 47/61; A61K 45/06

(52) U.S. Cl. CPC A61K 47/61; A61K 45/06

(57) ABSTRACT

Described herein are compositions such as antibody-drug conjugates that include a heparosan polymer. In some embodiments, a targeting moiety is bound to an end of the heparosan polymer or to an internal monomeric subunit of the heparosan polymer. In some embodiments, a payload molecule is bound to an end of the heparosan or to an internal monomeric subunit. Some embodiments relate to methods of making and using the compositions.

### RELATED APPLICATIONS

This application claims the benefit of U.S. Provisional Application No. 62/689,582, filed Jun. 25, 2018, and of U.S. Provisional Application No. 62/689,553, filed Jun. 25, 2018. The contents of the aforementioned applications are expressly incorporated herein by reference in their entirety.

### STATEMENT REGARDING FEDERALLY SPONSORED RESEARCH OR DEVELOPMENT

This invention was made with government support under Contract Number R01 HL062244-05A1 awarded by the National Institutes of Health. The government has certain rights in the invention.

### FIELD

Described herein are heparosan compositions, and methods of making and using them. Some such embodiments may include an antibody-drug conjugate that include heparosan. Some such embodiments include a targeting moiety and/or payload molecule bound to an end of the heparosan or bound to an internal monomeric subunit of the heparosan.

### BACKGROUND

An antibody-drug conjugate (ADC) is an antibody linked to a payload to form an immunoconjugate. The antibody may specifically bind to a target cell, which often results in the ADC being internalized by the cell so that a treatment drug is released into, and treats, the cell. Because ADCs can specifically target a cell, side effects are reduced compared to systemically administering the drug that is not linked to an antibody. While antibodies and their derivatives (e.g., Fab, Fab′, single chain Ab, nanobodies) are commonly used, other non-immunoglobulin sequence proteins (e.g., lectins, matrix adherins, receptors) that selectively bind a target species or ligand also fall into the scope of this principle. In a similar vein, polynucleotides with affinity for a target (e.g., aptamers) may also be utilized instead of an antibody.

### SUMMARY

Some embodiments of the compositions and methods described herein relate to a conjugate. In some embodiments, the conjugate includes: a heparosan polymer having a first end, a second end and internal monomeric subunits; a targeting moiety bound to the first end of the heparosan polymer; and a first payload molecule bound to the second end or an internal monomeric subunit of the heparosan polymer.

Some embodiments relate to a method of producing a conjugate. In some embodiments, the method includes: reacting a heparosan synthase with UDP-GlcA and UDP-GlcNAc and a functionalized primer to create a heparosan polymer intermediate with a reactive end; contacting the reactive end of the intermediate with at least one targeting moiety to attach the targeting moiety to the heparosan polymer intermediate; reacting the heparosan polymer intermediate with a heparosan synthase, a functionalized UDP-precursor, and at least one of UDP-GlcA and UDP-GlcNAc to create a reactive heparosan polymer; and contacting the reactive heparosan polymer with at least one payload molecule to form a conjugate.

In some embodiments, the method includes: reacting a heparosan polymer with reductive amination to create a reactive heparosan polymer intermediate having a reactive end; contacting the reactive end of the intermediate with at least one targeting moiety to attach the targeting moiety to the heparosan polymer intermediate; reacting the heparosan polymer intermediate with at least one activating reagent to create an active heparosan polymer; and contacting the active heparosan polymer with at least one payload molecule to form a conjugate.

### BRIEF DESCRIPTION OF THE DRAWINGS

FIG. 1 is a diagram that depicts one embodiment of heparosan functioning as a linker to attach payloads, including cytotoxic agents, therapeutic agents, or drugs to an antibody to create an antibody drug conjugate (ADC). The left side depicts attaching payloads to a heparosan backbone by linking the payloads to functionalities of the internal heparosan sugar rings that compose the heparosan chain. The right side depicts payloads being attached to specific terminal ends on the heparosan chain. Although many sugar chains are shown, embodiments described herein also include constructs with one heparosan chain per immunoglobulin-derived polypeptide.

FIG. 2 is a diagram that depicts one embodiment of defined sizes of heparosan polymers being simultaneously attached to both of the at least one antibody and a payload (herein denoted as “Y”).

FIG. 3 is a diagram that depicts one embodiment of the structure of the backbone of heparosan (depicted with various chain sizes) attached to an antibody, which allows for internal attachment of payloads (herein denoted as “X”) to the backbone.

### DETAILED DESCRIPTION

One embodiment disclosed herein is a conjugate comprising a targeting moiety, such as an antibody, bound to an end of a heparosan polymer. A payload molecule is then bound to an internal monomeric subunit of the heparosan polymer such that the entire composition provides a targeted therapeutic for delivering the payload to a specific site in the body.

Another embodiment disclosed herein is a conjugate comprising a targeting moiety bound to a first end of a heparosan polymer, but wherein the payload molecule is bound to a second end of the heparosan polymer instead of an internal monomeric subunit. In some embodiments, the targeting moiety is an antibody or a binding fragment thereof. In other embodiments, the targeting moiety comprises a polypeptide. In yet further embodiments, the targeting moiety comprises a polynucleotide.

In some embodiments, the payload molecule comprises a toxin. In some embodiments, the payload molecule comprises a therapeutic agent. In some embodiments, the payload molecule comprises a chemotherapeutic agent.

Another embodiment is a conjugate comprising a targeting moiety bound to a first end of a heparosan polymer; a payload molecule bound to a second end of the heparosan polymer; and a payload molecule bound to an internal monomeric subunit of the heparosan polymer.

One embodiment is a conjugate that comprises a targeting moiety linked to a payload through the terminal ends of a heparosan (“HEP”) linker. In this embodiment, the targeting moiety may be any molecule that can bind to another molecule. For example, a targeting moiety may include an antibody or its fragments, a T-cell receptor, chimeric antibody molecule, or an affinity reagent.

The payload may be a toxic agent, including chemotoxic agents, therapeutic agents, chemotherapeutic agents, peptides, proteins, small molecule toxins, or other toxic agents. In this embodiment, the linker is the sugar polymer heparosan (HEP). Discussed below is a suite of sugar chemoenzymatic synthesis and manufacture tools to both control chain length and chemical composition of heparosan linker molecules.

Another embodiment is a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with a heparosan synthase, a functionalized UDP-precursor, and at least one of UDP-GlcA and UDP-GlcNAc to create a reactive heparosan polymer wherein the heparosan polymer can be reacted within the heparosan chain with at least one payload molecule, wherein the at least one payload molecule is attached along a backbone of the heparosan polymer.

Certain embodiments are directed to a method of producing a targeting moiety-heparosan polymer-payload molecule conjugate, wherein the method comprises the step of reacting, either simultaneously or wholly or partially sequentially, the heparosan polymer produced by the method of the immediately preceding paragraph with at least one targeting moiety and at least one payload molecule, thereby producing the targeting moiety-heparosan polymer-payload molecule conjugate in which the at least one targeting moiety is attached to a first end of the heparosan polymer and the at least one payload molecule is attached along a backbone of the heparosan polymer.

Another embodiment is a method of producing a carrier to construct a targeting moiety-heparosan polymer-payload molecule conjugate. The method includes the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with a heparosan synthase, a functionalized UDP-precursor, and at least one of UDP-GlcA and UDP-GlcNAc to create a reactive heparosan polymer wherein the heparosan polymer can be reacted at or near its non-reducing terminus with at least one payload molecule, wherein the at least one payload molecule is attached to a second end of the heparosan polymer.

Another embodiment is directed to a method of producing a targeting moiety-heparosan polymer-payload molecule conjugate, wherein the method comprises the step of reacting, either simultaneously or wholly or partially sequentially, the heparosan polymer carrier produced by the method described in the immediately preceding paragraph with at least one targeting moiety and at least one payload molecule, thereby producing the targeting moiety-heparosan polymer-payload molecule conjugate in which the at least one targeting moiety is attached to a first end of the heparosan polymer and the at least one payload molecule is attached to a second end of the heparosan polymer.

Certain embodiments are directed to a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with a heparosan synthase and a functionalized UDP-precursor to create a reactive heparosan polymer wherein the heparosan polymer can be reacted within the heparosan chain with at least one payload molecule, wherein the at least one payload molecule is attached along a backbone of the heparosan polymer.

Certain embodiments are directed to a method of producing a targeting moiety-heparosan polymer-payload molecule conjugate, wherein the method comprises the step of reacting, either simultaneously or wholly or partially sequentially, the heparosan polymer produced by the method of the immediately preceding paragraph with at least one targeting moiety and at least one payload molecule, thereby producing the targeting moiety-heparosan polymer-payload molecule conjugate in which the at least one targeting moiety is attached to a first end of the heparosan polymer and the at least one payload molecule is attached along a backbone of the heparosan polymer.

Another embodiment is directed to a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one payload molecule, wherein the at least one payload molecule is attached to a first end of the heparosan polymer; and (b) reacting in a second step the heparosan polymer intermediate with a heparosan synthase, a functionalized UDP-precursor, and at least one of UDP-GlcA and UDP-GlcNAc to create a reactive heparosan polymer wherein the heparosan polymer can be reacted at or near its non-reducing terminus with at least one targeting moiety, wherein the at least one targeting moiety is attached to a second end of the heparosan polymer.

Another embodiment is directed to a method of producing a targeting moiety-heparosan polymer-payload molecule conjugate, wherein the method comprises the step of reacting, either simultaneously or wholly or partially sequentially, the heparosan polymer carrier produced by the method described in the immediately preceding paragraph with at least one targeting moiety and at least one payload molecule, thereby producing the targeting moiety-heparosan polymer-payload molecule conjugate in which the at least one payload molecule is attached to a first end of the heparosan polymer and the at least one targeting moiety is attached to a second end of the heparosan polymer.

Certain embodiments are directed to a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; (b) reacting in a second step the heparosan polymer intermediate with at least one activating reagent to create a reactive heparosan polymer wherein the heparosan polymer can be reacted within the heparosan chain with at least one payload molecule, wherein the at least one payload molecule is attached along a backbone of the heparosan polymer; and (c) reacting the reactive heparosan polymer with at least one payload molecule to attach the at least one payload molecule along a backbone of the heparosan polymer.

Certain embodiments of the above method further comprise the step of reacting the reactive heparosan polymer having the at least one payload molecule attached along a backbone thereof with at least one targeting moiety to produce the targeting moiety-heparosan polymer-payload molecule conjugate.

Yet another embodiment is directed to a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the targeting moiety is attached to a first end of the heparosan polymer; and (b) reacting in a second step the heparosan polymer intermediate with a heparosan synthase and a functionalized UDP-precursor to create a reactive heparosan polymer wherein the heparosan polymer can be reacted at or near its non-reducing terminus with at least one payload molecule, wherein the payload molecule is attached to a second end of the heparosan polymer.

Another embodiment is directed to a method of producing a targeting moiety-heparosan polymer-payload molecule conjugate, wherein the method comprises the step of reacting, either simultaneously or wholly or partially sequentially, the heparosan polymer carrier produced by the method described in the immediately preceding paragraph with at least one targeting moiety and at least one payload molecule, thereby producing the targeting moiety-heparosan polymer-payload molecule conjugate in which the at least one targeting moiety is attached to a first end of the heparosan polymer and the at least one payload molecule is attached to a second end of the heparosan polymer.

Certain embodiments are directed to a method of producing a carrier to construct a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with at least one payload molecule to attach the at least one payload molecule along a backbone of the heparosan polymer.

Certain embodiments of the above method further comprise the step of reacting the reactive heparosan polymer having the at least one payload molecule attached along a backbone thereof with at least one targeting moiety to produce the targeting moiety-heparosan polymer-payload molecule conjugate.

Another embodiment is directed to a method of producing a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with a targeting moiety or a payload molecule, wherein the targeting moiety/payload molecule is attached to a first end of the heparosan polymer; (b) reacting in a second step the heparosan polymer intermediate with at least one activating reagent to create a reactive heparosan polymer wherein the heparosan polymer can be reacted at its non-reducing terminus with the other of the targeting moiety or payload molecule, wherein the other of the targeting moiety or payload molecule is attached along a backbone of the heparosan polymer; and (c) reacting, either simultaneously or wholly or partially sequentially, the reactive heparosan polymer with at least one payload molecule and at least one targeting moiety to produce the targeting moiety-heparosan polymer-payload molecule conjugate.

Another embodiment is directed to a method of producing a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with at least one payload molecule to attach the at least one payload molecule at a non-reducing terminus of the heparosan polymer.

In certain embodiments, the method above further comprises the step of reacting the reactive heparosan polymer having the at least one payload molecule attached to the non-reducing terminus thereof with at least one targeting moiety to produce the targeting moiety-heparosan polymer-payload molecule conjugate.

Certain embodiments are directed to a method of producing a carrier to construct a targeting moiety-heparosan polymer-payload molecule conjugate, wherein the method comprises the steps of: (a) reacting in a first step an isolated heparosan polymer with reductive amination to create a reactive heparosan polymer intermediate, wherein the heparosan polymer can be reacted at its reducing terminus with at least one targeting moiety, wherein the at least one targeting moiety is attached to a first end of the heparosan polymer; (b) reacting in a second step the heparosan polymer intermediate with at least one activating reagent to create a reactive heparosan polymer wherein the heparosan polymer can be reacted within the heparosan chain with at least one payload molecule, wherein the at least one payload molecule is attached along a backbone of the heparosan polymer; and (c) reacting the reactive heparosan polymer with at least one payload molecule to attach the at least one payload molecule along a backbone of the heparosan polymer.

Certain embodiments of the above method further comprise the step of reacting the reactive heparosan polymer having the at least one payload molecule attached along a backbone thereof with at least one targeting moiety to produce the targeting moiety-heparosan polymer-payload molecule conjugate.

Certain embodiments are directed to a method of producing a carrier to construct a targeting moiety-heparosan polymer-payload molecule conjugate, wherein the method comprises the steps of: (a) reacting in a first step an isolated heparosan polymer with reductive amination to create a reactive heparosan polymer intermediate, wherein the heparosan polymer can be reacted at its reducing terminus with at least one targeting moiety, wherein the at least one targeting moiety is attached to a first end of the heparosan polymer; and (b) reacting in a second step the heparosan polymer intermediate with at least one payload molecule to attach the at least one payload molecule along a backbone of the heparosan polymer.

Certain embodiments of the above method further comprise the step of reacting the reactive heparosan polymer having the at least one payload molecule attached along a backbone thereof with at least one targeting moiety to produce the targeting moiety-heparosan polymer-payload molecule conjugate.

Another embodiment is directed to a method of producing a targeting moiety-heparosan polymer-payload molecule conjugate, wherein the method comprises the steps of: (a) reacting in a first step a heparosan synthase with UDP-GlcA and UDP-GlcNAc and a functionalized primer to create a reactive heparosan polymer intermediate, wherein the heparosan polymer can be reacted at its reducing terminus with at least one payload molecule, wherein the at least one payload molecule is attached to a first end of the heparosan polymer; and (b) reacting in a second step the heparosan polymer intermediate with at least one targeting moiety to attach the at least one targeting moiety at a non-reducing terminus of the heparosan polymer.

In certain embodiments, the method above further comprises the step of reacting the reactive heparosan polymer having the at least one targeting moiety attached to the non-reducing terminus thereof with at least one payload molecule to produce the targeting moiety-heparosan polymer-payload molecule conjugate.

Another embodiment is directed to a method of producing a targeting moiety-heparosan polymer-payload molecule conjugate, wherein the method comprises the steps of: (a) reacting in a first step an isolated heparosan polymer with reductive amination to create a reactive heparosan polymer intermediate, wherein the heparosan polymer can be reacted at its reducing terminus with at least one of a targeting moiety and a payload molecule, wherein the targeting moiety or payload molecule is attached to a first end of the heparosan polymer; (b) reacting in a second step the heparosan polymer intermediate with at least one activating reagent to create a reactive heparosan polymer wherein the heparosan polymer can be reacted at its non-reducing terminus with the other of the targeting moiety or payload molecule, wherein the other of the targeting moiety or payload molecule is attached along a backbone of the heparosan polymer; and (c) reacting, either simultaneously or wholly or partially sequentially, the reactive heparosan polymer with at least one payload molecule and at least one targeting moiety to produce the targeting moiety-heparosan polymer-payload molecule conjugate.

Another embodiment is directed to a method of producing a targeting moiety-heparosan polymer-payload molecule conjugate, wherein the method comprises the steps of: (a) reacting in a first step an isolated heparosan polymer with reductive amination to create a reactive heparosan polymer intermediate, wherein the heparosan polymer can be reacted at its reducing terminus with at least one of a targeting moiety and a payload molecule, wherein the targeting moiety or payload molecule is attached to a first end of the heparosan polymer; and (b) reacting, either simultaneously or wholly or partially sequentially, the reactive heparosan polymer with at least one payload molecule and at least one targeting moiety to produce the targeting moiety-heparosan polymer-payload molecule conjugate.

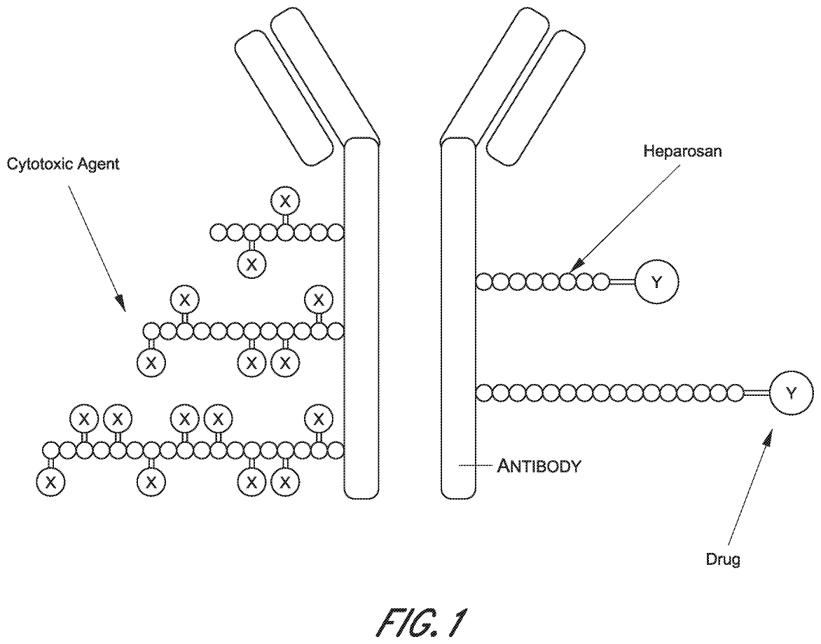

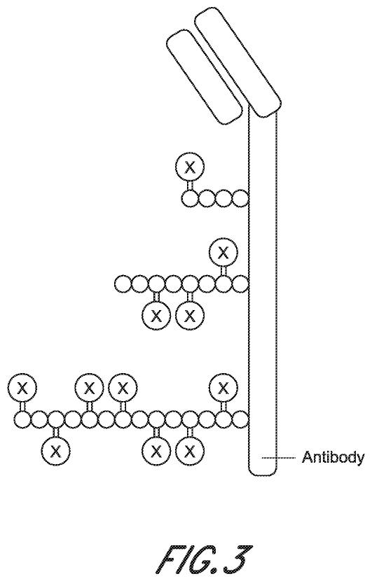

In embodiments related to the cases described above, the use of the HEP chain to carry multiple payloads along the backbone allows more drug or toxin molecules to be delivered in a given conjugate molecule (FIGS. 1 and 3). Therefore, the effect of less potent molecules may be amplified and rendered more effective by the use of a HEP chain carrier.

### Definitions

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “heparosan” as used herein will be understood to refer to a carbohydrate chain with a repeat structure of [-4-glucuronic acid-1-beta-4-N-acetylglucosamine-1-alpha-]ₙ, (also referred to as [-4-GlcA-1-β-4-GlcNAc-1-α-]ₙ), wherein n is 1 or greater. In certain non-limiting examples, n may be from about 2 to about 10,000. The term “oligosaccharide” generally denotes n being from about 1 to about 11, while the term “polysaccharide” denotes n being equal to or greater than 12. The term “heparosan” may be utilized interchangeably with the terms “N-acetylheparosan” and “unsulfated, unepimerized heparin.”

The term “UDP-sugar” as used herein refers to a carbohydrate modified with uridine diphosphate (e.g., UDP-N-acetylglucosamine).

The term “conjugate” as used herein refers to a complex created between two or more compounds by covalent or non-covalent bonds. The term “covalent” as used herein refers to the sharing of electrons between atoms to create a chemical interaction.

The term “antibody” (or “Ab”) herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5th Ed., Garland Publishing, New York). An antibody can be of any type or class (e.g., IgG, IgE, IgM, IgD, and IgA) or subclass (e.g., IgG1, IgG2, IgG3, IgG4, IgA1, and IgA2). The antibody can be derived from any suitable species. In some embodiments, the antibody is of human or murine origin. An antibody can be, for example, human, humanized, or chimeric.

An “antibody fragment” comprises a portion of an intact antibody, such as (but not limited to) the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immuno specifically bind to a target antigen (e.g., a cancer cell antigen, a viral antigen, or a microbial antigen).

The term “specific binding” refers to antibody binding to a predetermined antigen. Typically, the antibody binds with an affinity of at least about 1×10⁷ M⁻¹, and binds to the predetermined antigen with an affinity that is at least two-fold greater than its affinity for binding to a non-specific antigen (e.g., BSA, casein) other than the predetermined antigen or a closely-related antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

The term “monoclonal antibodies” specifically includes “chimeric” antibodies in which a portion of the heavy and/or light chain is identical to or homologous with the corresponding sequence of antibodies derived from a particular species or belonging to a particular antibody class or subclass, while the remainder of the chain(s) is identical to or homologous with the corresponding sequences of antibodies derived from another species or belonging to another antibody class or subclass, as well as fragments of such antibodies, so long as they exhibit the desired biological activity.

As used herein, “isolated” means separated from other components of (a) a natural source, such as a plant or animal cell or cell culture, or (b) a synthetic organic chemical reaction mixture.

As used herein, “purified” means that when isolated, the isolate contains at least 95%, and in another aspect at least 98%, of a compound (e.g., a conjugate) by weight of the isolate.

Some embodiments include a therapeutically effective amount of a composition as described herein such as an ADC. The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and potentially stop) cancer cell infiltration into peripheral organs; inhibit (i.e., slow to some extent and potentially stop) tumor metastasis; inhibit, to some extent, tumor growth; and/or relieve to some extent one or more of the symptoms associated with the cancer. To the extent the drug may inhibit the growth of and/or kill existing cancer cells, it may be cytostatic and/or cytotoxic. For cancer therapy, efficacy can, for example, be measured by assessing the time to disease progression (UP) and/or determining the response rate (RR).

Some embodiments include a pharmaceutical composition comprising a composition described herein such as an ADC. A “pharmaceutical composition” refers to an agent that may be administered in vivo to bring about a therapeutic and/or prophylactic/preventative effect.

Some embodiments include a treatment with a composition described herein such as an ADC. The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder.

Some embodiments include a method of treat cancer with a composition described herein such as an ADC. In the context of cancer, the term “treating” includes any or all of inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody; lessening the autoimmune-antibody burden; and ameliorating one or more symptoms of an autoimmune disease.

The terms “loading” or “drug loading” or “payload loading” represent or refer to the average number of payloads (“payload” and “payloads” are used interchangeable herein with “drug” and “drugs”) per targeting moiety or antibody in an ADC molecule. Drug loading may range from 1 to 20 drugs per antibody. This is sometimes referred to as the DAR, or drug to antibody ratio. Compositions of the ADCs described herein typically have DAR'S of from 1-20, and in certain embodiments from 1-8, from 2-8, from 2-6, from 2-5, and from 2-4. Typical DAR values are 2, 4, 6, and 8. The average number of drugs per antibody, or DAR value, may be characterized by conventional means such as UV/visible spectroscopy, mass spectrometry, ELISA assay, and HPLC. The quantitative DAR value may also be determined. In some instances, separation, purification, and characterization of homogeneous ADCs having a particular DAR value may be achieved by means such as reverse phase HPLC or electrophoresis. DAR may be limited by the number of attachment sites on the antibody. These embodiments broadly include: (i) restricted number of available chemical functionalities/groups of a desired class or use, (ii) induction of molecular aggregation or insolubility that lowers clinical usefulness or manufacturing/regulatory requirements, and/or (ii) biochemical issues that lower or destroy antibody (or other polypeptide) activities or its native state(s).

Some embodiments include a composition described herein such as an ADC or a pharmaceutically acceptable salt thereof. The term “pharmaceutically acceptable salt” refers to salts that retain the biological effectiveness and properties of a compound and which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; and in particular, include the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Subject,” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate.

### Utilities and Applications:

Some embodiments include a method of treating a patient for a disease by administering an active agent-conjugate as disclosed and described herein to said patient. In some embodiments, the patient may have cancer, an autoimmune disease, or diabetes.

Some embodiments include a method of diagnosis or imaging by administering an active agent-conjugate as disclosed and described herein to an individual.

### Embodiments of Structures:

Some embodiments provide a multi-functional linker comprising heparosan or a pharmaceutically acceptable salt thereof. Heparosan comprises a polymer formed from repeating monomeric units:

[-4-GlcA-1-beta-4-GlcNAc-1-alpha-]ₙ

wherein n represents the number of each monomeric unit;

wherein n may be between 2 and 10,000;

wherein each monomeric unit may comprise four hydroxyl groups;

wherein every other monomeric unit may comprise a carboxylate group;

wherein GlcNAc is N-acetylglucosamine;

wherein certain derivatives may possess a free amino group; and

wherein GlcA is glucuronic acid.

In some embodiments, the conjugate composition comprises a targeting moiety linked to a payload, such as a cytotoxic agent or a therapeutic agent, wherein the targeting moiety is linked to the payload using heparosan. In some embodiments, the payload is attached to an internal monomeric unit within heparosan. In some embodiments, the payload is attached to a hydroxyl group on a monomeric unit. In some embodiments, the payload is attached to a carboxylate group on a monomeric unit. In addition, the native heparosan may be further derivatized (e.g., to form a free amino group, etc.) or an artificial heparosan may be synthesized with additional or non-native functional groups (e.g., to add an azido or alkyne group, etc.) to allow the payload to be attached.

In some embodiments, a conjugate composition is provided comprising a targeting moiety linked to more than one payload, wherein the targeting moiety is linked to the more than one payload using heparosan polymers. In some embodiments, the more than one payload is bound to internal monomers within the heparosan polymers. In some embodiments, the more than one payload is bound to hydroxyl groups on the monomeric subunits. In some embodiments, the more than one payload is bound to carboxylate groups on the monomeric subunits. In some embodiments, the more than one payload is bound to both hydroxyl groups and carboxylate groups on the monomeric subunits.

In some embodiments, two or more payloads are attached to adjacent monomers within the heparosan polymer. In some embodiments, two or more payloads are attached to non-adjacent monomers within the heparosan polymer. In some embodiments, between about 1 and about 10,000 non-adjacent monomers have payload molecules attached thereto. In other examples, between about 1 and about 1,000, between about 5 and about 1,000, between about 5 and about 150, between about 5 and about 100, between about 5 and about 50, and between about 10 and about 25 non-adjacent monomers have payload molecules attached thereto.

### Modifications to the Heparosan Chain:

In some embodiments, the heparosan chain is (1) modified at one end (e.g., either the reducing and/or non-reducing termini) to attach to the targeting moiety and (2) the payload is attached to the backbone groups at one or multiple sites of the heparosan chain. Due to its ability to be made in a variety of polymer sizes, even to vary long chain lengths (10s, 100s, or 1000s of sugar units, or molecular weights (MW) of many kDa or several MDa), heparosan may be used to add a predetermined number of payload molecules to each heparosan chain attached to a targeting moiety.

The two native (i.e., naturally existing) sites for modification on the heparosan chain are the (a) carboxylic acid (COOH; derived from the 1 GIcA per disaccharide repeat) and (b) hydroxyl (OH) functionalities. With pre-treatment (e.g., base treatment, hydrazinolysis) to remove the natural acetyl (Ac) or unnatural (trifluoroAc=TFA) group that is coupled to the glucosamine (GlcN), a third site, (c) the amine functionality (NH₂), is unmasked and available for payload molecules to be covalently linked to the heparosan chain.

### HEP/Pavload Coupling Reactions:

There are many ways of coupling a payload molecule, such as a cytotoxic agent, to the heparosan backbone, depending on the chemistry of the payload molecule.

A. For carboxylate groups (COOH) on a payload molecule, hydrazides can be used. The hydrazides have pKa values between 2 and 4, are nucleophilic at pH 4.8 and couple efficiently to carbodiimide-activated glucuronic acid (GlcA) residues of heparosan. In some embodiments, the use of dihydrazide compounds, such as adipic dihydrazide (ADH), will yield multiple hydrazide groups for further derivatization with a payload molecule (i.e., one end of ADH attached to heparosan and the other to the payloads). The activated payload (e.g., taxol-NHS ester) is then loaded onto the polymer.

In some embodiments, the mechanism of coupling the hydrazide to heparosan occurs through the mechanism below:

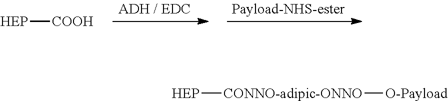

In some embodiments, the reaction of heparosan with a large excess of an amine-containing payload at pH 6.8 in the presence of a soluble carbodiimide and 1-hydroxybenzotriazole (HOBt) in aqueous dimethylsulfoxide (DMSO) allows coupling.

B. For hydroxyl groups (OH) on a payload molecule, payload molecules with an amino group (e.g., doxorubicin, daunomycin, etc.) may be coupled to heparosan via cyanogen bromide (CNBr) activation. In some embodiments, a reaction scheme activates the OH groups of the monosaccharides to a highly-reactive isourea intermediate. The drug is attached via a urethane bond to one of the hydroxylic functions of the heparosan.

In some embodiments, the mechanism of coupling the amino group to heparosan occurs through the mechanism below:

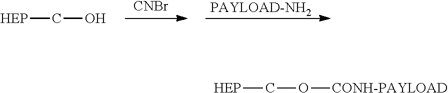

C. For amino groups (NH₂; unmasked from heparosan backbone amides) on a payload molecule, payload molecules with NHS-esters or similar amino-reactive moieties may be directly coupled to the heparosan chain in aqueous buffers at neutral pH. In some embodiments, hydrazine or a strong base is used on natural heparosan or the use of artificial heparosan with GlcN[TFA] sugars treated with milder base to expose useful amounts of free amine. The new amine can be used to add payload to the HEP-chain, either directly (e.g., via NHS-ester route, etc.) or indirectly (i.e., convert into an intermediate that then reacts with the payload in a later step).

In some embodiments, the mechanism of coupling NHS-esters to heparosan occurs through the mechanism below:

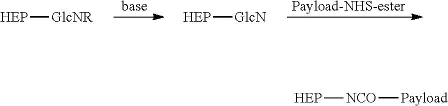

### Payload Release:

Depending on the chemistry needed for the payload and its desired target in the body, the heparosan-payload linkage may be stable (i.e., payload not released for action) or unstable (i.e., payload-heparosan bond is cleaved after administration). The latter bonds may be triggered to break by pH changes (e.g., low pH that can exist in some tissues such as inflamed areas or some cell compartments such as the lysosome) or via actions of endogenous enzymes (e.g., esterases, proteases, etc.) or simply slow release (e.g., moderately stable self-immolative linkers).

### Heparosan Size Control:

The size of the chemoenzymatically synthesized heparosan chain may be either (I) controlled by step-wise sugar addition with heparosan synthases (<^{˜}3-20 sugar units) and/or (II) by synchronized, stoichiometrically controlled synthesis (>10-20 sugar units). In some embodiments, the heparosan synthase enzyme, acceptor, and UDP-sugars are combined in the various combinations, order of addition, and molar ratios in the appropriate reaction buffers (neutral aqueous solution with divalent cations) to yield molecules with ^{˜}3-4 sugar units to ^{˜}10,000 units, as desired.

In some embodiments, the resulting HEP size distribution can be very narrow and thus referred to as monodisperse (i.e., population with identical chain sizes) or quasi-monodisperse (i.e., population with very similar chain lengths). Typically, the step-wise elongation synthesis of process (I) above yields monodisperse HEP polymers with a polydispersity index (M_{w}/Mₙ where M_{w} is the weight-average molar mass, and Mₙ is the number-average molar mass) approaching 1, the ideal case. Typically, the synchronized polymerization process of (II) above yields quasi-monodisperse HEP polymers with a polydispersity index of 1.002-1.2.

In some embodiments, in a process (III), the acceptor is omitted from the above polymerization reaction, and the UDP-sugars participate in de novo synthesis (i.e., a hydroxyl group of the monosaccharide unit of one UDP-sugar itself serves as the acceptor group that is then elongated by the addition a second monosaccharide from another UDP-sugar); however, the size control is lost without the synchronization effect mediated by the acceptor (i.e., bypass the rate-limiting step of biosynthesis, initiation; the elongation with subsequent sugars is much faster). Therefore, these acceptor-less reactions of process (III) will yield more polydisperse (i.e., wide size distribution) HEP chains. In addition, the acceptor can add an extra range of chemical functionality beyond normal native carbohydrate chemistry (e.g., add an additional handle such as amine, aldehyde, etc.).

In some embodiments, in a process (IV), polysaccharides derived from bacterial fermentation of microbes (e.g., Escherichia coli K5 or Pasteurella multocida Type D or recombinant strains with the heparosan synthesizing enzymes) are a source of heparosan. In some embodiments, these microbial preparations are even more polydisperse than the three chemoenzymatic processes (I)-(III) described above. A further aspect of these preparations derived from microbes is that the spent culture media or encapsulated cells are much more complicated mixtures (including, in some cases, endotoxins or cell wall fragments) than the chemoenzymatic systems (which employ very defined starting materials such as purified enzyme, isolated sugars, simple chemical reagents, etc.); thus, more effort must be expended to reach purities required for conjugation syntheses and/or patient administration.

### Heparosan Handle Addition:

All linear polysaccharides (and most carbohydrates) possess directionality that is similar to the ‘amino’ and ‘carboxyl’ termini of proteins; the sugar chain has a ‘reducing end’ (i.e., the site of maturation of the terminal sugar that creates an aldehyde or ‘reducing’ sugar) and a ‘non-reducing end.’ Disclosed herein are various glycosaminoglycan (GAG) syntheses where chemical functionalities or handles can be added or built at either or both ends of the chain.

### Targeting Moieties:

As used herein, the term “targeting moiety” refers to a structure that binds or associates with a biological moiety or fragment thereof.

In some embodiments, the targeting moiety may be an antibody. In some embodiments, the targeting moiety may be a monoclonal antibody (mAb). In some embodiments, the targeting moiety may be an antibody fragment, surrogate, or variant. In some embodiments, the targeting moiety may be a protein ligand. In some embodiments, the targeting moiety may be a protein scaffold. In some embodiments, the targeting moiety may be a peptide. In some embodiments, the targeting moiety may be RNA or DNA. In some embodiments, the targeting moiety may be a RNA or DNA fragment. In some embodiments, the targeting moiety may be a small molecule ligand.

In some embodiments, the targeting moiety may be an antibody fragment such as (but not limited to) those described in Janthur et al. (“Drug Conjugates Such as Antibody Drug Conjugates (ADCs), Immunotoxins and Immunoliposomes Challenge Daily Clinical Practice,” Int. J. Mol. Sci. (2012), 13: 16020-16045, the disclosure of which is incorporated herein by reference in its entirety). In some embodiments, the targeting moiety may be an antibody fragment such as (but not limited to) those described in Trail, PA (“Antibody Drug Conjugates as Cancer Therapeutics,” Antibodies (2013), 2: 113-129, the disclosure of which is incorporated herein by reference in its entirety).

In some embodiments, the targeting moiety may be HuM195-Ac-225, HuM195-Bi-213, Anyara (naptumomab estafenatox; ABR-217620), AS1409, Zevalin (ibritumomab tiuxetan), BIIB015, BT-062, Neuradiab, CDX-1307, CR011-vcMMAE, Trastuzumab-DM1 (R3502), Bexxar (tositumomab), IMGN242, IMGN388, IMGN901, ¹³¹I-labetuzumab, IMMU-102 (⁹⁰Y-epratuzumab), IMMU-107 (⁹⁰Y-clivatuzumab tetraxetan), MDX-1203, CAT-8015, EMD 273063 (hu14.18-IL2), Tucotuzumab celmoleukin (EMD 273066; huKS-IL2), ¹⁸⁸Re-PTI-6D2, Cotara, L19-IL2, Teleukin (F16-IL2), Tenarad (F16-¹³¹I), L19-¹³¹I, L19-TNF, PSMA-ADC, DI-Leu16-IL2, SAR3419, SGN-35, or CMC544. In some embodiments, the targeting moiety may comprise, consist of, or consist essentially of the antibody portion of HuM195-Ac-225, HuM195-Bi-213, Anyara (naptumomab estafenatox; ABR-217620), AS1409, Zevalin (ibritumomab tiuxetan), BIIB015, BT-062, Neuradiab, CDX-1307, CR011-vcMMAE, Trastuzumab-DM1 (R3502), Bexxar (tositumomab), IMGN242, IMGN388, IMGN901, ¹³¹I-labetuzumab, IMMU-102 (⁹⁰Y-epratuzumab), IMMU-107 (⁹⁰Y-clivatuzumab tetraxetan), MDX-1203, CAT-8015, EMD 273063 (hu14.18-IL2), Tucotuzumab celmoleukin (EMD 273066; huKS-IL2), ¹⁸⁸Re-PTI-6D2, Cotara, L19-IL2, Teleukin (F16-IL2), Tenarad (F16-¹³¹I), L19-¹³¹I, L19-TNF, PSMA-ADC, DI-Leu16-IL2, SAR3419, SGN-35, or CMC544.

In some embodiments, the targeting moiety may be Brentuximab vedotin, Trastuzumab emtansine, Inotuzumab ozogamicin, Lorvotuzumab mertansine, Glembatumumab vedotin, SAR3419, Moxetumomab pasudotox, Moxetumomab pasudotox, AGS-16M8F, AGS-16M8F, BIIB-015, BT-062, IMGN-388, or IMGN-388.

In some embodiments, the targeting moiety may comprise, consist of, or consist essentially of the antibody portion of Brentuximab vedotin, Trastuzumab emtansine, Inotuzumab ozogamicin, Lorvotuzumab mertansine, Glembatumumab vedotin, SAR3419, Moxetumomab pasudotox, Moxetumomab pasudotox, AGS-16M8F, AGS-16M8F, BIIB-015, BT-062, IMGN-388, or IMGN-388.

In some embodiments, the targeting moiety may comprise, consist of, or consist essentially of Brentuximab, Inotuzumab, Gemtuzumab, Milatuzumab, Trastuzumab, Glembatumomab, Lorvotuzumab, or Labestuzumab.

### Peptides and Amino Acids:

In some embodiments of the methods and compositions described herein, the ADC comprises a peptide. Some embodiments include a peptide. In some embodiments, the peptide, such as the antibody, is PEGylated. PEGylation can provide, for example, increased stability and/or efficacy of the polypeptide. Methods for PEGylation known in the art can be used in the methods and compositions provided herein. Such methods include, but are not limited to, those provided in Khalili et al., (“Comparative Binding of Disulfide-Bridged PEG-Fabs,”Bioconjugate Chemistry (2012), 23(11): 2262-2277; Cong et al., (“Site-Specific PEGylation at Histidine Tags,” Bioconjugate Chemistry (2012), 23(2): 248-263; Brocchini et al. (“Disulfide bridge based PEGylation of proteins,” Advanced Drug Delivery Reviews (2008), 60(1): 3-12; Balan et al. (“Site-Specific PEGylation of Protein Disulfide Bonds Using a Three-Carbon Bridge,” Bioconjugate Chemistry (2007), 18(1): 61-76; Shaunak et al. (“Site-specific PEGylation of native disulfide bonds in therapeutic proteins,” Nature Chemical Biology (2006), 2(6): 312-313; and Godwin et al. (“PEG derivative conjugated proteins and peptides for use in pharmaceuticals,” WO 2010/100430). All of the aforementioned PEGylation references are incorporated by reference in their entireties.

Heparosan (HEP) is also useful as a “PEG substitute” because it is more biocompatible then PEG (DeAngelis P L. Expert Opinions in Drug Delivery (2015), 12: 349-352); these natural sugars found in all animals from the protozoa Hydra to humans are recognized as ‘self’ and can be degraded in the lysosomes when their task is complete. In some embodiments, the artificial PEG polymer can be immunogenic and accumulate in the body since it is recognized as not being ‘self,’ and there is no efficient, non-toxic degradation pathway in humans. Therefore, an additional attribute of HEP is that it can improve the stability or efficacy or pharmacodynamics of the various conjugates described in the present disclosure.

Certain commonly encountered amino acids which provide useful substitutions for the active agent-conjugates include, but are not limited to, β-alanine (β-Ala) and other omega-amino acids such as 3-aminopropionic acid, 2,3-diaminopropionic acid (Dpr), 4-aminobutyric acid, and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); naphthylalanine (Nal); 4-phenylphenylalanine, 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe(pNH₂)); N-methyl valine (MeVal); homocysteine (hCys), homophenylalanine (hPhe), and homoserine (hSer); hydroxyproline (Hyp), homoproline (hPro), N-methylated amino acids, and peptoids (N-substituted glycines).

Other amino acid residues not specifically mentioned herein can be readily categorized based on their observed physical and chemical properties in light of the definitions provided herein.

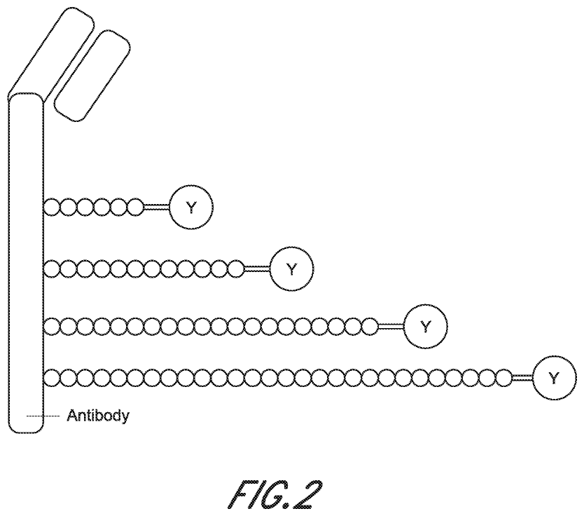

In some embodiments, antibody drug conjugates are provided as depicted in FIGS. 1, 2, and 3. In FIGS. 1, 2, and 3, heparosan is used to link a cytotoxic agent or a drug to an antibody. The left side of FIG. 1 and FIG. 3 depict cytotoxic agents bound to internal monomeric units within heparosan. The right side of FIG. 1 and FIG. 2 depict drugs bound to terminal units on heparosan. In some embodiments, the cytotoxic agents link to carboxylate groups on the monomeric units of heparosan. In some embodiments, the cytotoxic agents link to hydroxyl groups on the monomeric units of heparosan. In some embodiments, the cytotoxic agents link to newly exposed or artificially added groups on the monomeric units of heparosan.

### EXAMPLES

Examples are provided herein below. However, the presently disclosed embodiments are to be understood to not be limited in its application to the specific experimentation, results and laboratory procedures. Rather, the Examples are simply provided as one of various embodiments and are meant to be exemplary, not exhaustive.

Heparosan is a natural molecule found in the body. It is relatively biologically inert in the extracellular spaces (i.e., not significantly bound, degraded, or cleared in mammals), but will be degraded in the lysosomes after entry into cells.

Heparosan, in the simplest configuration, has two component coupling sites per sugar chain that can be used independently or in combination (e.g., Payload A on 1 end, and another Payload B on opposite end). If tri-molecular complexes are required for a medical application, then extra handles are introduced using hetero- or homo-trifunctional activating reagents. Multiplexing or ‘piggy-backing’ different molecules requires more planning, but is not usually limited until yields detrimentally influence the purity of the final target (e.g., accumulation of failure products reduces the percentage of target beyond an acceptable threshold).

The hydrophilic (water-loving) heparosan chain can be used to counterbalance solubility issues of the proteins. The negative charge also helps prevent aggregation, which is a big problem for many biologics.

The size of the heparosan chain can be controlled via its synthesis; thus, different linker spacings can be created (i.e., longer chain, more space between components), and/or the hydrodynamic size can be modified to alter the construct's pharmacodynamics (e.g., prevent renal filtration, induce steric hindrance to slow receptor-mediated or protease-mediated clearance, etc.).

Heparosan has multiple payload coupling sites per sugar chain that can be used independently or in combination (e.g., payload molecule A on COOHs, while payload molecule B on OHs, etc.).

The hydrophilic (water-loving) heparosan chain can be used to counterbalance solubility issues of the protein and/or the payload. The negative charge (if not fully modified) of HEP also helps prevent aggregation, a big problem for many biologics.

### Example 1: Reducing End Chemistry

Using an acceptor that is elongated by a GAG (glycosaminoglycan) synthase at the non-reducing end with new sugars from UDP-sugar precursors, a long chain defined heparosan is made where every polymer has any desired chemical functionality introduced on the acceptor located at the reducing end. Thus, an acceptor with an amino, sulfhydryl, or other reactive molecule can be employed in the synthesis of a like modified polymer. There will be only one added chemical group/chain that allows for precision linker design and use. In addition, the original chemical group in the sugar precursor may be transformed into a new functionality by derivatization chemistry known in the art.

### Example 2: Non-Reducing End Chemistry

Using the appropriate UDP-sugar analog (i.e., an artificial molecule that mimics the natural precursor and is incorporated into a chain, but also has a new chemical group) that is added by a GAG synthase onto the non-reducing end of an acceptor, a heparosan chain is made where the polymer has a chosen chemical functionality introduced by the analog located at the non-reducing end. Thus, an analog with an amino, sulfhydryl, azide, or other reactive molecule can be employed in the synthesis of a like modified polymer. If the step-wise synthesis route is employed, then there will be only one added chemical group/chain that is essential for precision linker design and use. If more payloads are desired, then more than one analog can be added by repeated step-wise addition and/or synchronized polymerization with a bigger pool of analog.

Alternatively, a protected (hidden) chemical group can also be added as an analog, then in a post-polymerization step, the protecting group is removed, thereby unmasking the once hidden useful group. The use of UDP-GlcN[TFA] to add a free amine group at the desired location is a way to gently add a new reactive group. Accordingly, after sugar addition and purification, the polymer intermediate is treated with mild base (e.g., volatile combination of triethylamine, methanol, water), lyophilized, then the material is ready to use in a linker reaction. In addition, the original chemical group may be transformed into a new functionality by derivatization chemistry already known in the art before linking to the target moieties (e.g., macromolecules, biologics, drugs, etc.).

### Example 3: Modifying the Heparosan Chain at the Reducing Termini and the Non-Reducing Termini

A heparosan chain that is (1) modified at one end (e.g., the reducing termini) is used to attach an antibody and (2) modified at the other end (e.g., the non-reducing termini) is used to attach to a toxin molecule. Due to its ability to be made in a variety of polymer sizes, even to very long chain lengths (10s, 100s, 1,000s of sugar units, heparosan may be used to link 2 target components), modifying the heparosan chain at the reducing termini and the non-reducing termini may be used to link an antibody (Ab; e.g., an IgG or Fab to cancer antigen, etc.) to a toxin (Tox; e.g., diphtheria toxin, ricin, etc.) component to create:

Tox-Non-reducing end-HEP-Reducing end-Ab=Tox-N-HEP-R-Ab.

The linker is made and deployed in a stepwise process using various GAG chemoenzymatic synthesis methods.

In this case, the steps are:

1. An acceptor with the amine functionality (NH₂) (e.g., GlcA-GlcNAc-GlcA-NH₂=Hep₃-NH₂) is reacted with a PmHS1 synthase catalyst and normal UDP-sugars (1-40 mM each UDP-GlcA and UDP-GlcNAc) in 20-50 mM Hepes (or other buffer), pH 7.2, with 1-10 mM MnCl₂ (or MgCl₂) to elongate (note: other steps employing a synthase may need buffers and cations, but for brevity, these reagents are assumed to be present elsewhere) and thus create the needed size final polymer=HEP-NH₂. The polymer is purified by anion exchange chromatography (e.g., Q Sepharose with NaCl gradient, pH 7.2) and concentrated by ultrafiltration against water.

2. The HEP-NH₂ is further step-wise elongated with 1.2-2 molar equivalents UDP-GlcA (to ensure that all sugar chains possess a non-reducing termini with GIcA) and PmHS1, and the resulting GlcA-HEP-NH₂ is re-purified as above (to avoid Step 2, a slight excess of UDP-GlcA may alternatively be used in Step 1).

3. The GlcA-HEP-NH₂ is step-wise elongated with UDP-GlcNAc-6-azide (all sugar chain end at the non-reducing termini with GlcNAc-6-azide [a ‘clickable’ functionality with alkynes; Otto et al., J. Biol. Chem. (2012) 287(1): 7203-12]) and PmHS1. The resulting polymer, GlcNAc-6-azide-GlcA-HEP-NH₂ is purified.

4. The GlcNAc-6-azide-GlcA-HEP-NH₂ is converted to GlcNAc-6-azide-GlcA-HEP-maleimide using an excess of NHS-maleimide reagent (creates a sulfhydryl-reactive end; AMAS [N-α-maleimidoacet-oxysuccinimide ester] or SMPH [Succinimidyl 6-((beta-maleimidopropionamido)hexanoate); or a use reagent that introduces a new iodoacetamide or pyridylthio, etc.]; Thermo Fisher) in 50 mM Hepes or phosphate (any non-amine buffers), pH 7-7.5 buffer. The polymer is purified by gel filtration chromatography.

5. The linker is now ready for coupling the two components: (a) reduced antibody (e.g., Fab with a free thiol; reacts with maleimide); and (b) an alkyne-tagged toxin (e.g., toxin treated with 3-Propargyloxypropanoic Acid, Succinimidyi Ester; Thermonsher). First, the reduced, desalted Ab is added to the linker in neutral pH buffer (50 mM Hepes or phosphate, pH 7-7.5) for 1-3 hours. Second, the purified alkyne-toxin and the ‘click’ catalyst (copper (II) sulfate in the presence of a reductant such as ascorbic acid to generate copper (I)) is added (note: however, if a cyclic strained alkyne reagent is employed, then no copper is required). After 1-3 hours, the final target, Tox-HEP-Ab, is purified by anion exchange and gel filtration chromatography steps along with sterile filtration before use in the patient.

### Example 4: Converting Heparosan Polymers Into Various Chemical Functionalities

Heparosan polymers can be converted into various chemical functionalities using the appropriate NHS-ester (or similar) to introduce a variety of handles, including but not limited to:

Heparosan Handle (Reactivity)

Aldehyde (amine)

Squarane (amine)

Alkyne (azide)

Azide (alkyne)

Chelator (metals)

Thiol (maleimide, iodoacetyl)

Maleimide, lodoacetyl (thiol).

### Example 5: Breaking Unstable Bonds of the Hep/Drug Linkage

Depending on the chemistry needed for the linker and its desired action on a certain compartment or tissue in the body, the HEP-payload linkage may be stable (component not released for action; described here) or unstable (component-heparosan bond is cleaved; using alternative activating reagents with labile bonds as known in the art). The latter bonds may be triggered to break by pH changes (e.g., low pH that can exist in some tissues such as inflamed areas or cell compartments like the lysosome) or via actions of endogenous enzymes (e.g., esterases, proteases, etc.) or simply slow release (e.g., moderately stable self-immolative linkers).

### Example 6: Incorporating an Amine Group

An alternative non-reducing end handle that may be introduced into a heparosan chain is the amine group via a scheme with the unnatural analog UDP-GlcN[TFA] (trifluoroAc=TFA). The PmHS2 heparosan synthase enzyme (and chimeric derivatives) will incorporate the UDP-sugar either singly or multiple times. After the unnatural sugar is incorporated and the polmyer is purified, the TFA group is removed from the glucosamine (GlcN) to expose the amine functionality (NH₂) using a mild base (e.g., volatile triethylamine/methanol/water mixture) that does not affect the normal Ac present on all the other GlcNAc residues on the heparosan chain. The unmasked amine handle is then available for further molecules to be coupled (a macromolecular component directly, or after the use of an amine-reactive NHS-ester heterobifunctional reagent to introduce other groups such as maleimide, azide, alkyne, aldehyde, etc. that are later used as handles for linking components).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A conjugate, comprising:
a heparosan polymer having a first end, a second end and internal monomeric subunits;
a targeting moiety bound to the first end of the heparosan polymer; and
a first payload molecule bound to the second end or an internal monomeric subunit of the heparosan polymer.

2. The conjugate of claim 1, wherein the targeting moiety comprises an antibody or binding fragment thereof.

3. The conjugate of claim 1, wherein the targeting moiety comprises a polypeptide.

4. The conjugate of claim 1, wherein the targeting moiety comprises a polynucleotide.

5. The conjugate of claim 1, wherein the first payload molecule comprises a toxin.

6. The conjugate of claim 1, wherein the first payload molecule comprises a therapeutic agent.

7. The conjugate of claim 1, wherein the first payload molecule comprises a chemotherapeutic agent.

8. The conjugate of claim 1, wherein the first payload molecule is bound to the internal monomeric subunit, and further comprising a second payload molecule bound to the second end of the heparosan polymer

9. The conjugate of claim 8, wherein the conjugate comprises two or more of the first payload molecules bound to internal monomeric subunits of the heparosan polymer.

10. The conjugate of claim 1, wherein the first payload molecule is bound to the second end of the heparosan polymer.

11. A method of producing a conjugate, comprising:
reacting a heparosan synthase with UDP-GlcA and UDP-GlcNAc and a functionalized primer to create a heparosan polymer intermediate with a reactive end;
contacting the reactive end of the intermediate with at least one targeting moiety to attach the targeting moiety to the heparosan polymer intermediate;
reacting the heparosan polymer intermediate with a heparosan synthase, a functionalized UDP-precursor, and at least one of UDP-GlcA and UDP-GlcNAc to create a reactive heparosan polymer; and
contacting the reactive heparosan polymer with at least one payload molecule to form a conjugate.

12. A method of producing a conjugate, comprising:
reacting a heparosan polymer with reductive animation to create a reactive heparosan polymer intermediate having a reactive end;
contacting the reactive end of the intermediate with at least one targeting moiety to attach the targeting moiety to the heparosan polymer intermediate;
reacting the heparosan polymer intermediate with at least one activating reagent to create an active heparosan polymer; and
contacting the active heparosan polymer with at least one payload molecule to form a conjugate.

13. The method of claim 11, wherein the at least one payload molecule is attached along a backbone of the heparosan polymer.

14. The method of claim 11, wherein the wherein the heparosan polymer is reacted at or near its non-reducing terminus with at least one payload molecule so that the at least one payload molecule is attached to a second end of the heparosan polymer.

15. The method of claim 12, wherein the active heparosan polymer is reacted at or near its non-reducing terminus with at least one payload molecule so that the at least one payload molecule is attached to a second end of the heparosan polymer.

16. The method of claim 11, wherein reacting a heparosan synthase, contacting the reactive end of the intermediate, and contacting the reactive heparosan polymer with at least one payload molecule is performed in the same reaction step.

17. The method of claim 11, wherein reacting a heparosan synthase, contacting the reactive end of the intermediate, and contacting the reactive heparosan polymer with at least one payload molecule is performed in different reaction steps.

18. The method of claim 11, wherein the targeting moiety comprises an antibody or binding fragment thereof.

19. The method of claim 11, wherein the targeting moiety comprises a polypeptide.

20. The method of claim 11, wherein the targeting moiety comprises a polynucleotide.

21. The method of claim 11, wherein the payload molecule comprises a toxin.

22. The method of claim 11, wherein the payload molecule comprises a therapeutic agent.

23. The method of claim 11, wherein the payload molecule comprises a chemotherapeutic agent.
